(19) United States
(12) Patent Application Publication
(10) Pub. No.: US 2005/0075634 A1
(43) Pub. Date: Apr. 7, 2005

(54) Interspinous process implant with radiolucent spacer and lead-in tissue expander

(76) Inventors: James F. Zucherman, San Francisco, CA (US); Ken Y. Hsu, San Francisco, CA (US); Charles J. Winslow, Walnut Creek, CA (US); John Flynn, Concord, CA (US); Steve Mitchell, Pleasant Hill, CA (US)

Correspondence Address: Sheldon R. Meyer;FLIESLER DUBB MEYER & LOVEJOY LLP, Fourth Floor, Four Embarcadero Center, San Francisco, CA, 94111-4156, US

(21) Appl. No.: 10/694,103

(22) Filed: Oct. 27, 2003

Related U.S. Application Data
(60) Provisional application No. 60/421,915, filed on Oct. 29, 2002.

(51) Int. Cl. A61B 17/56

(52) U.S. Cl. 606/61

(57) ABSTRACT

The present invention is directed to an interspinous process device with a deflectable spacer which can be placed between adjacent spinous processes to limit the movement of the vertebrae. The device limits the range of motion of the spinous processes. The spacer and a lead-in distraction guide or tissue expander can be radiolucent.

CLAIM TO PRIORITY

This application claims priority to U.S. Provisional Application No. 60/421,915, filed Oct. 29, 2002, entitled “INTERSPINOUS PROCESS IMPLANT WITH RADIOLUCENT SPACER AND LEAD-IN TISSUE EXPANDER,” which is incorporated herein by reference.

### CROSS-REFERENCE TO RELATED APPLICATIONS

This application is related to U.S. patent application Ser. No. 10/230,505, filed Aug. 29, 2002, entitled “DEFLECTABLE SPACER FOR USE AS AN INTERSPINOUS PROCESS IMPLANT AND METHOD,” U.S. Provisional Application No. 60/421,921, filed Oct. 29, 2002, entitled “INTERSPINOUS PROCESS APPARATUS AND METHOD WITH A SELECTABLY EXPANDABLE SPACER,” and U.S. patent application Ser. No. 10/______ filed Oct. 14, 2003, entitled “INTERSPINOUS PROCESS APPARATUS AND METHOD FOR SELECTABLY EXPANDABLE SPACER,” which are incorporated herein by reference. This application is also related to U.S. patent application Ser. No. 10/037,236, filed Nov. 9, 2001, which is related to U.S. patent application Ser. No. 09/799,215, filed Mar. 5, 2001, which is related to U.S. patent application Ser. No. 09/473,173, filed Dec. 28, 1999, now U.S. Pat. No. 6,235,030, which is related to U.S. patent application Ser. No. 09/179,570, filed October 27, 1998, now U.S. Pat. No. 6,048,342, which is related to U.S. patent application Ser. No. 09/474,037, filed Dec. 28, 1999, now U.S. Pat. No. 6,190,387, which is related to U.S. patent application Ser. No. 09/175,645, filed Oct. 20, 1998, now U.S. Pat. No. 6,068,630. All of the above are incorporated herein by reference.

### FIELD OF THE INVENTION

This invention relates to an interspinous process implant.

### BACKGROUND OF THE INVENTION

The spinal column is a bio-mechanical structure composed primarily of ligaments, muscles, vertebrae and intervertebral disks. The bio-mechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

As the present society ages, it is anticipated that there will be an increase in adverse spinal conditions which are characteristic of older people. By way of example, with aging comes an increase in spinal stenosis (including, but not limited to, central canal and lateral stenosis), and facet arthropathy. Spinal stenosis typically results from the thickening of the bones that make up the spinal column and is characterized by a reduction in the available space for the passage of blood vessels and nerves. Pain associated with such stenosis can be relieved by medication and/or surgery. Of course, it is desirable to eliminate the need for major surgery for all individuals, and, in particular, for the elderly.

In addition, there are a variety of other ailments that can cause back pain in patients of all ages. For these ailments it is also desirable to eliminate such pain without major surgery.

Accordingly, there needs to be developed implants for alleviating such conditions which are minimally invasive, can be tolerated by patients of all ages, and, in particular, the elderly, and can be performed preferably on an out patient basis.

### SUMMARY OF THE INVENTION

The present invention is directed to providing a minimally invasive implant for alleviating discomfort associated with the spinal column. The implant is characterized in one embodiment in that the spacer and the lead-in tissue expander or distraction guide are comprised of a material that is radiolucent. In another embodiment, the spacer can be deflectable. Suitable materials include, for example, polyetheretherketone (PEEK) and polyetherketoneketone (PEKK). Other material that can be used include polyetherketone (PEK), polyetherketoneetherketoneketone (PEKEKK), and polyetheretherketoneketone (PEEKK), and, generally, a polyaryletheretherketone. Further, other polyketones can be used as well as other thermoplastics. Such materials are advantageously radio-translucent, radiolucent or transparent to x-rays or other imaging techniques. Additional suitable materials can be selected from the groups including by way of example, high molecular weight polymers, and thermoplastics. Thus, the radiolucent nature of the spacer and distraction guide enables the implant to retain a high degree of structural support after being implanted while not impairing the ability to view the patient's anatomy in a subsequent x-ray. Other aspects, objects, features and elements of embodiments of the invention are described or evident from the accompanying specification, claims and figures.

### BRIEF DESCRIPTION OF THE DRAWINGS

FIGS. 1A-1F. FIG. 1A is a front plan view of an embodiment of an assembled implant of the invention; FIG. 1B is a left side view of the embodiment of the invention of FIG. 1A; FIG. 1C is a front plan view of the embodiment of the invention of FIG. 1A including a spacer, a main body and a first wing; FIG. 1D is a left side view of the second wing of the embodiment of the invention of FIG. 1A; FIG. 1E is a front plan view of the second wing of the embodiment of the invention of FIG. 1A; FIG. 1F is an end view of the spacer of the embodiment of the invention of FIG. 1A.

FIG. 2A is a perspective view of an embodiment of the frame of the tissue expander or distraction guide of the invention. FIG. 2B is a perspective view of an embodiment of the lead-in tissue expander or distraction guide of the invention.

FIGS. 3A and 3B are an end and a perspective view of still another embodiment of the spacer of the invention. FIG. 3C is a front view of the spacer of FIG. 3A.

FIGS. 4A and 4B are an end and a perspective view of yet another embodiment of the spacer of the invention.

FIGS. 5A and 5B are an end and a perspective view of still another embodiment of the spacer of the invention.

FIGS. 6A and 6B are an end and a perspective view of a further embodiment of the spacer of the invention.

### DETAILED DESCRIPTION OF THE PREFERRED EMBODIMENTS OF THE INVENTION

The following description is presented to enable any person skilled in the art to make and use the invention. Various modifications to the embodiments described will be readily apparent to those skilled in the art, and the principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all patents and patent applications cited in this application are incorporated herein by reference

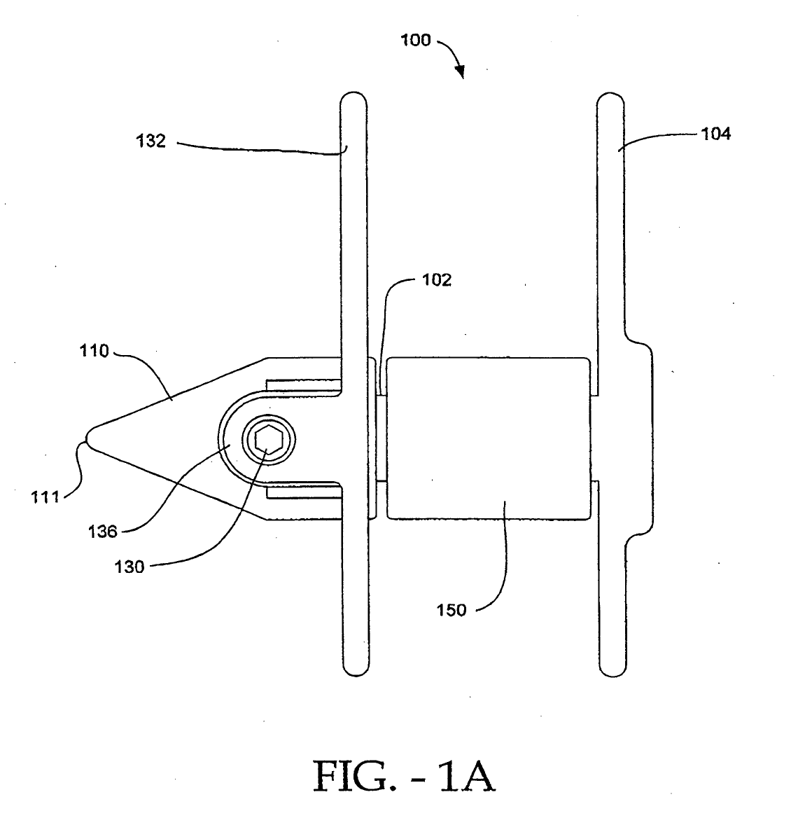

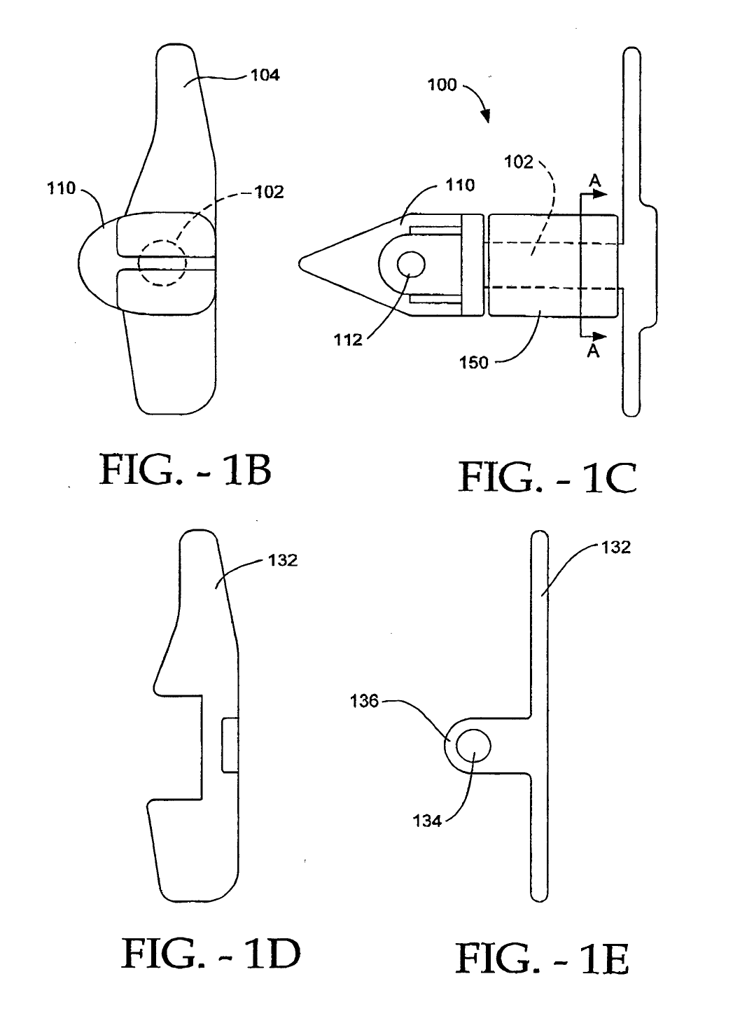

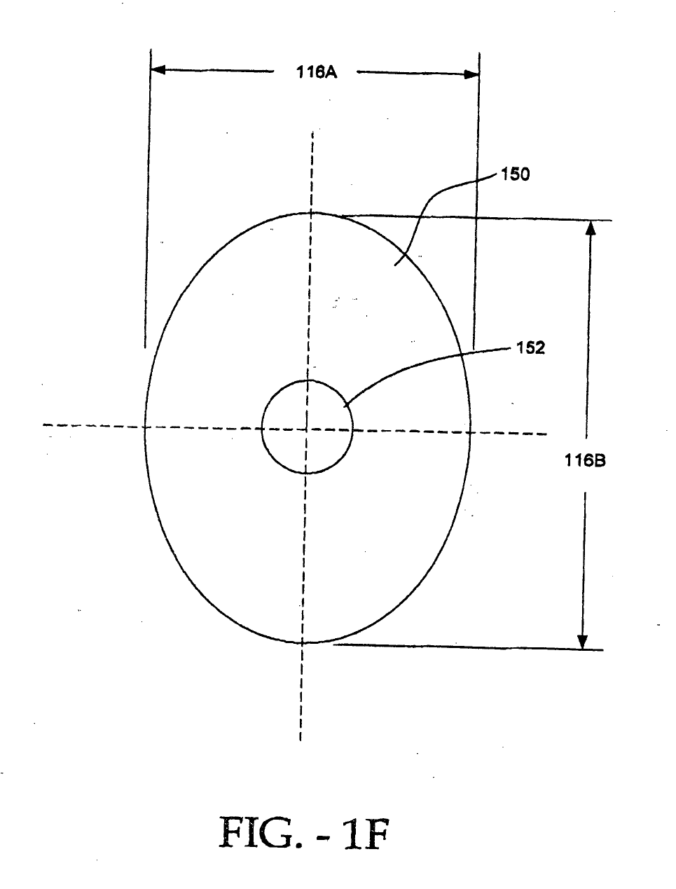

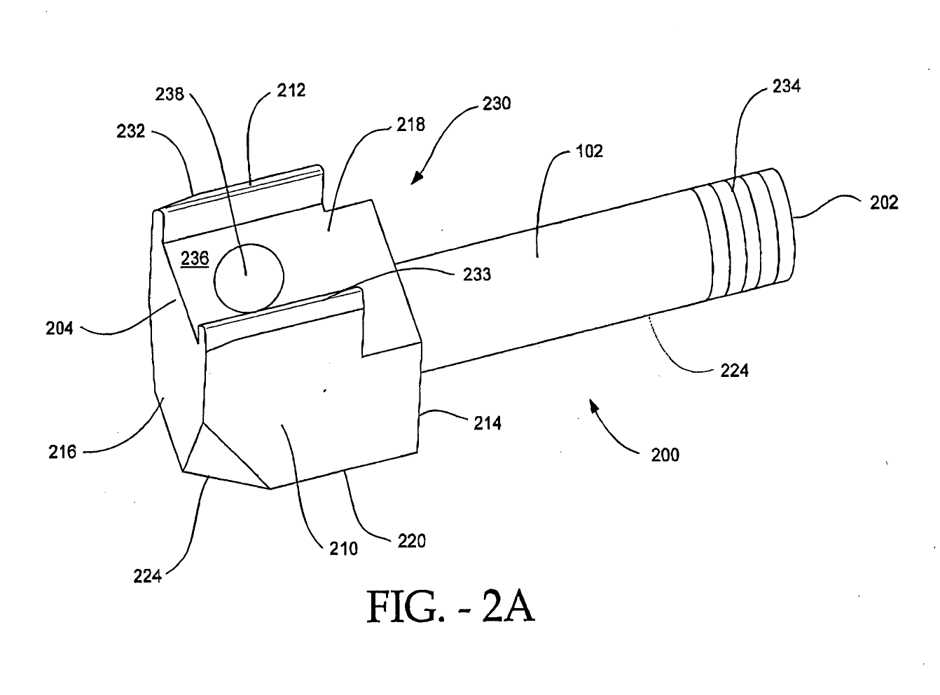

An embodiment of an implant 100 of the invention is depicted in FIG. 1A. This implant 100 includes a first wing 104 and a spacer 150 and a lead-in tissue expander or distraction guide 110. This embodiment further can include, as required, a second wing 132. As can be seen in FIG. 1A, a shaft 102 extends from the first wing 104 and is the body that connects the first wing 104 to the tissue expander or distraction guide 110. Also, as can be seen in FIGS. 1A and 1B, the distraction guide 110 in this particular embodiment acts to distract the soft tissue and the spinous processes when the implant 100 is inserted between adjacent spinous processes. In this particular embodiment, the guide 110 has an expanding cross-section from the distal end 111 to the area where the second wing 132 is secured to the guide 110. In this embodiment the guide 110 is wedge-shaped.

Additionally, as can be seen in FIGS. 1A and 1F, the spacer 150 is elliptical-shaped in cross-section. The spacer 150 can have other shapes such as circular, oval, ovoid, football-shaped, and rectangular-shaped with rounded corners and other shapes, and be within the spirit and scope of the invention. In this preferred embodiment, the spacer 150 includes a bore 152 which extends the length of the spacer 150. The spacer 150 is received over the shaft 102 of the implant 100 and can rotate thereon about the shaft 102. In these embodiments, the spacer 150 can have minor and major dimensions as follows:

| Minor Dimension (116a) | Major Dimension (116 b) |
|---|---|
| 6 mm | 13.7 mm |
| 8 mm | 14.2 mm |
| 10 mm | 15.2 mm |
| 12 mm | 16.3 mm |
| 14 mm | 17.8 mm |

The advantage of the use of the spacer 150 as depicted in the embodiment of FIG. 1A, is that the spacer 150 can be rotated and repositioned with respect to the first wing 104, in order to more optimally position the implant 100 between spinous processes. It is to be understood that the cortical bone or the outer bone of the spinous processes is stronger at an anterior position adjacent to the vertebral bodies of the vertebra than at a posterior position distally located from the vertebral bodies. Also, biomechanically for load bearing, it is advantageous for the spacer 150 to be close to the vertebral bodies. In order to facilitate this and to accommodate the anatomical form of the bone structures, as the implant is inserted between the spinous processes and/or urged toward the vertebral bodies, the spacer 150 rotates relative to the wings, such as wing 104, so that the spacer 150 is optimally positioned between the spinous processes, and the wing 104 is optimally positioned relative to the spinous processes. Further, the broad upper and lower surfaces of the spacer 150 helps spread the load that the spinous processes place on the spacer 150.

As may be required for positioning the implant 100 between the spinous processes, the implant 100 can also include a second wing 132 which fits over the guide 110 and is secured by a bolt 130 placed through an aperture 134 provided in a tongue 136 of second wing 132. The bolt 130 is received and secured in the threaded bore 112 located in the guide 110. As implanted, the first wing 104 is located adjacent to first sides of the spinous processes and the second wing 132 is located adjacent to second sides of the same spinous processes.

In another embodiment, the spacer 150 has a cross-section with a major dimension and a minor dimension, wherein the major dimension is greater than the minor dimension, and, for example, less than about two times the minor dimension. It is to be understood that the spacer 150 can be fabricated from somewhat flexible and/or deflectable material.

In this embodiment the spacer is made out of a polymer, more specifically, the polymer is a thermoplastic. Still more specifically, the polymer is a polyketone known as polyetheretherketone (PEEK). Still more specifically, the material is PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com). The spacer 150 can be formed by extrusion, injection, compression molding and/or machining techniques. This material has appropriate physical and mechanical properties and is suitable for carrying and spreading the physical load between the spinous process. Further in this embodiment, the PEEK has the following additional approximate properties:

| Property | Value | |
|---|---|---|
| Density | 1.3 | g/cc |
| Rockwell M | 99 | |
| Rockwell R | 126 | |
| Tensile Strength | 97 | MPa |
| Modulus of Elasticity | 3.5 | GPa |
| Flexural Modulus | 4.1 | GPa |

In a preferred embodiment, the implant 100 is comprised in part of titanium or other suitable implant material which may be radiopaque and in part of a radiolucent material that does not show up under x-ray or other type of imaging. In a preferred embodiment, the first and second wings and the shaft are comprised of such a radiopaque material such as titanium and the spacer and the distraction guide or tissue expander are comprised of a radiolucent material such as, for example, PEEK or PEKK or other radiolucent materials described herein. In an embodiment which includes the first wing, the spacer and the tissue expander, under imaging, the implant looks like an “T”. In an embodiment which includes both a first and a second wing, the spacer and the tissue expander, under imaging, the implant looks like a “H”. This embodiment allows the doctor to have a clearer view of the spine under imaging without the implant interfering as much with the view of the bone structure.

It should be noted that the material selected may also be filled. For example, other grades of PEEK are also available and contemplated, such as 30% glass-filled or 30% carbon-filled, provided such materials are cleared for use in implantable devices by the FDA, or other regulatory body. Glass-filled PEEK reduces the expansion rate and increases the flexural modulus of PEEK relative to that which is unfilled. The resulting product is known to be ideal for improved strength, stiffness, or stability. Carbon-filled PEEK is known to enhance the compressive strength and stiffness of PEEK and lower its expansion rate. Carbon-filled PEEK offers wear resistance and load carrying capability.

In this embodiment, as described above, the spacer 150 is manufactured from polyetheretherketone (PEEK), available from Victrex. As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. The spacer can also be comprised of polyetherketoneketone (PEKK).

Other material that can be used include polyetherketone (PEK), polyetherketoneetherketoneketone (PEKEKK), and polyetheretherketoneketone (PEEKK), and generally a polyaryletheretherketone. Further, other polyketones can be used as well as other thermoplastics. The spacer can also be made of titanium.

Reference to appropriate polymers that can be used in the spacer can be made to the following documents, all of which are incorporated herein by reference. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and, PCT Publication WO 02/00270 A1, dated Jan. 3, 2002, entitled “Bio-Compatible Polymeric Materials.”

Other materials such as Bionate®, polycarbonate urethane, available from the Polymer Technology Group, Berkeley, Calif., may also be appropriate because of the good oxidative stability, biocompatibility, mechanical strength and abrasion resistance. Other thermoplastic materials and other high molecular weight polymers can be used.

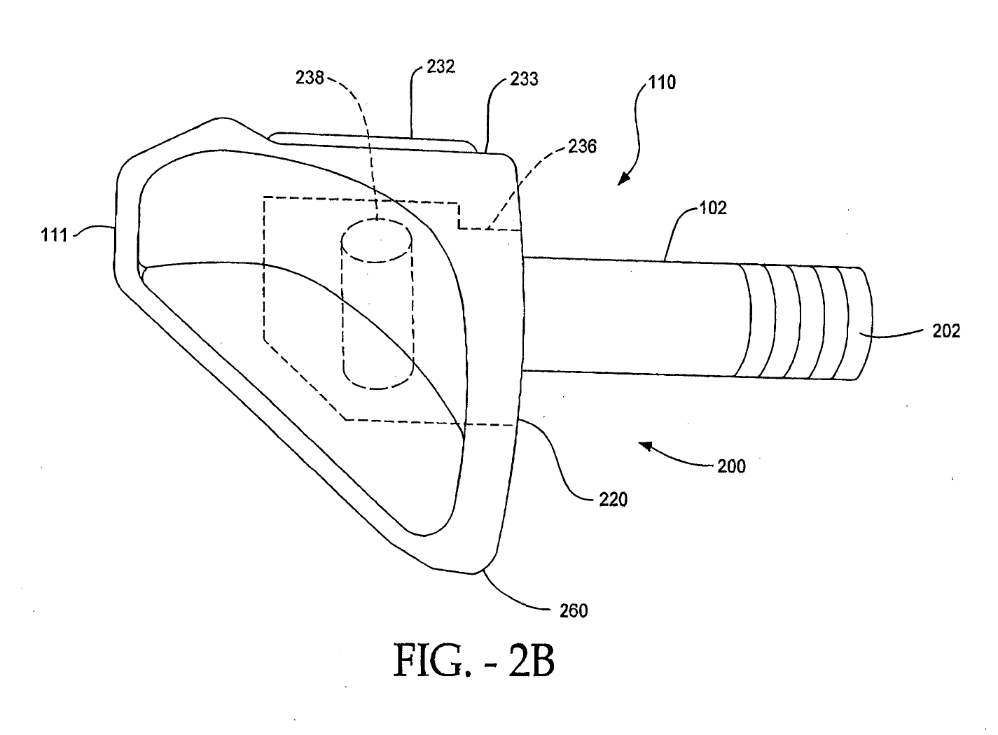

FIG. 2A and FIG. 2B shown an embodiment of the distraction guide or tissue expander 110. FIG. 2A shows a frame 200 for a distraction guide 110. The frame 200 is typically manufactured from radiopaque material such as titanium. The frame 200 has a first end 202 and a second end 204. The first end 202 has a shaft 102 which can be threaded with threads 234 at one end to facilitate connection to, for example, a first wing 104. The remaining end of the shaft connects to a distraction head frame 230 for the distraction guide 110. Alternatively, the shaft 102 and the distraction head frame 230 can be formed integral to each other.

Further, the distraction head frame 230, the shaft 102 and the first wing 104 can be formed as one unit. Still further in an embodiment with a screw thread 234 formed at one end of the shaft 102, which thread 234 is received in a threaded bore of the first wing 102, the thread 234 can be laser welded into the threaded bore of the first wing 102, if desired.

The distraction head frame 230 is formed to take on a relatively low profile because, as described above, it is typically formed of radiopaque material. As shown in FIG. 2A, distraction head frame 230 has two pairs of parallel sides. The first pair of parallel sides 210, 212 extends into a pair of flanges 232, 233 that define a recess 236. The second pair of parallel sides 214, 216 are perpendicular to the first pair of parallel sides. One of the second pair of parallel sides 214 abuts the shaft 102. As will be appreciated by those of skill in the art, neither the first or second pair of parallel sides need be parallel to each other, nor do the first pair of parallel sides need to be perpendicular to the second pair of parallel sides in order to practice the invention.

With respect to the frame 200 in FIG. 2A, the distraction head frame 230 has an upper surface 218 within the recess 236 with a threaded bore 112 therein. The threaded bore 112 receives, for example, a bolt 130 to secure the second wing 132 to the distraction guide 110 via the tongue 136 on the second wing 132 (shown in more detail with respect to FIG. 1A). The profile of the bolt 130 is such that the height of the bolt 130 and the tongue 136 fits within the recess 236.

The lower surface 220 opposing the upper surface 218 can have a first portion 222 that is parallel, or substantially parallel, to the upper surface 218. Additionally, a second portion 224 can be angled from the first portion 222 toward one of the second parallel sides 216. The angled configuration of the lower surface 220 is designed to facilitate the angled profile of the distraction guide.

FIG. 2B shows a perspective view of the distraction guide 110. The frame 200, as described above, is manufactured from radiopaque material. A cap 260 is formed of radiolucent material, such as a suitable polymer, around the frame 200. Suitable polymers include, but are not limited to the polyketones discussed above with respect to the spacer configurations. Accordingly, for example, PEEK, PEKK, PEK, PEKEKK and PEEKK can be used as well as the other materials that are suitable for the spacer 150. As will be appreciated by those of skill in the art, the cap 260 can be associated with the frame 200 by a variety of techniques such that the cap 260 is formed to the frame 200 or is adhered to the frame 200 using a suitable method. As illustrated in FIG. 2B, the cap 260 has a higher profile than the frame 200 and is shaped to facilitate the second end 204 of the distraction guide 110 acting to expand tissue when the distraction guide is implanted between spinous processes or used to distract adjacent spinous processes.

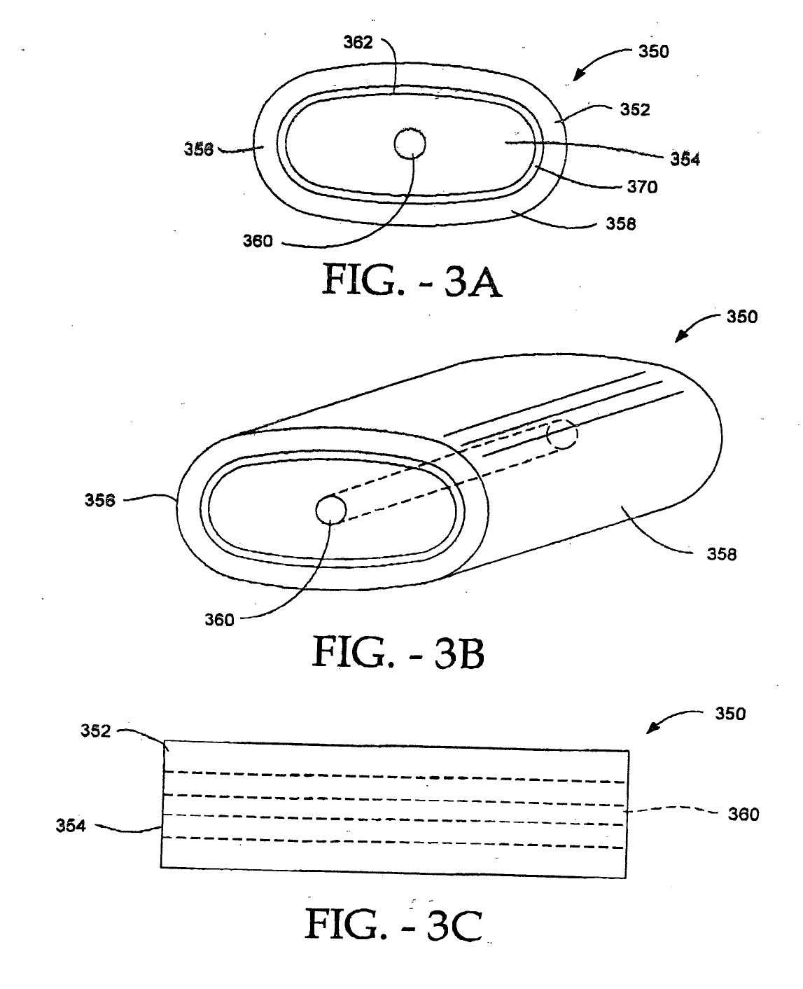

Referring now to FIGS. 3A-6B, various embodiments of spacers are depicted. In FIGS. 3A, 3B and 3C, the spacer 350 includes an outer spacer 352 and an inner spacer 354. Inner spacer 354 has a bore 360 therethrough that enables the spacer 350 to rotate about the shaft 102 of implant 100 shown in FIG. 1A.

Each of the inner and outer spacers of the spacer 350 can have a cross-section that is elliptical, oval, ovoid, football-shaped, circular-shaped, rectangular with rounded ends (where the cross-section has two somewhat flattened surfaces and two rounded surfaces similar to the effect of a flattened ellipse). Further, the inner spacer and outer spacer can have different cross-sectional shapes relative to each other. At least the minor outer diameter of the outer spacer is between 6 mm and 14 mm. Typically, the minor outer dimension is one of 6 mm, 8 mm, 10 mm, 12 mm, and 14 mm. The different sizes enable the spacer to accommodate different sized patients.

As depicted in FIG. 3A, the spacer 350 is a rectangle with rounded ends or a flattened ellipse, as it has two sides that are almost parallel to each other, and the ends connecting the parallel sides are curved, similar to a “race-track.” Thus, in this and other embodiments, the two sides or surfaces of the spacer, including the upper and the lower spacer, can also be flattened or slightly radiused. The bore 360 is located in the center of the inner spacer 354 and there is a gap 362 between the upper and lower portions of the outer spacer 352 and the inner spacer 354. A gap 370 is provided between the inner and outer spacers at the rounded ends 356, 358. In a preferred embodiment, for about an 8 millimeter spacer 350, the upper and lower gaps 362 are about 0.012 of an inch or about a quarter of a millimeter each for a total combined gap of about one half of a millimeter. The gaps 370 at the curved ends 356, 358 are about 0.002 of an inch or slightly less than a tenth of a millimeter each in a preferred embodiment. The gap 370 for all of the other spacers is preferably, as specified above, for the 8 mm spacer. For the 6 millimeter spacer, generally this is made of one piece such as seen in FIG. 1F. However, for the other spacers, these spacers are preferably made of two pieces as seen for example in FIG. 3A. The table below sets our preferred dimensions for the combined upper and lower gap dimension for the spacers.

| Spacer Minor Dimension | Total Combined Gap Dimension |
|---|---|
| 6 mm | n/a |
| 8 mm | 0.020 in (0.51 mm) |
| 10 mm | 0.025 in (0.64 mm) |
| 12 mm | 0.030 in (0.76 mm) |
| 14 mm | 0.035 in (0.89 mm) |

The gap 362 closed and the inner and outer spacers touch each other when the spacer is loaded with 800 newtons of force. The design is made to take repeated loading at 1200 newtons of force.

In the above embodiment, the outer spacer 352 is movably or slidably mounted on the inner spacer 354, and the inner spacer 354 is rotatably mounted on the shaft 102 of the implant 100.

As discussed above, the spacer, including either the inner spacer or outer spacer, or both, can be made of deflectable and flexible material. As discussed above, suitable material is a polymer such as for example polyetheretherketone (PEEK). Other suitable materials can include those described above. Further, titanium can be used.

Further, the deflectable or flexible material can have a graduated stiffness to help gradually distribute the load when the spinous processes place a force upon the exterior surface of the outer spacer 352. This can be accomplished by forming multiple layers of the deflectable or flexible material with decreasing stiffness or hardness from the center of the spacer 350 outwardly. Alternatively, the material can have a higher stiffness or hardness in the center of the inner spacer.

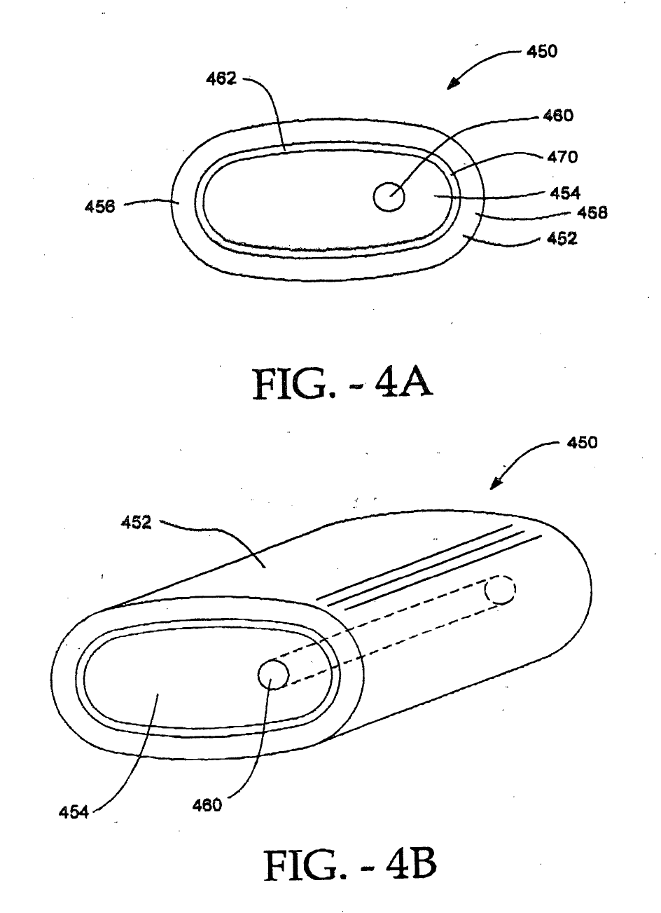

Persons of skill in the art will appreciate that the embodiments shown in FIGS. 4A-6B, can be made of the materials similar to those emphasized in the embodiment shown in FIGS. 1A and 3A.

Now referring to FIGS. 4A and 4B, again the spacer 450 is depicted as a somewhat flattened ellipse with rounded ends 456, 458, where two sides are somewhat parallel to each other and the ends connecting the parallel sides are curved, similar to a “race-track.” The bore 460 is located off-center within the inner spacer 454. Further, there are gaps 462, 470 between the outer spacer 452 and the inner spacer 454. Except for the location of the bore 460, the dimensions and materials of the embodiment of FIGS. 4A and 4B are similar to that of FIG. 3A and FIG. 3B.

The off-center bore 460 allows a greater portion of the spacer 450 to be positioned close to the vertebral bodies. With an ovoid (“egg-shaped”) spacer, off-set the bore 460 is preferably close to the bulbous end of the spacer with the more pointed end directed toward the vertebral bodies in order to attain the advantages of the spacer being closer to the vertebral bodies and enhanced distributed load bearing.

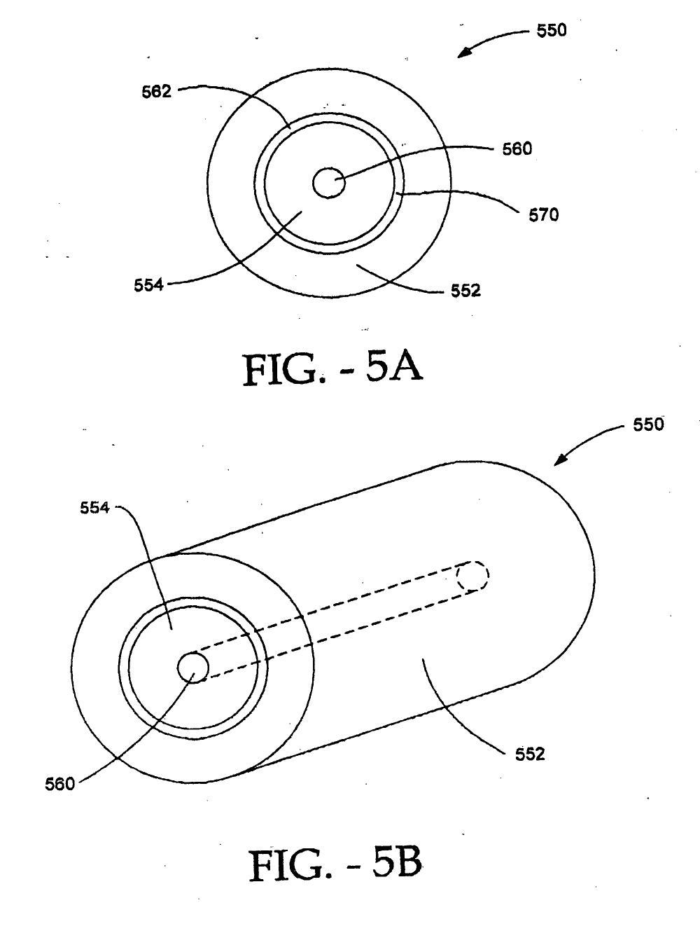

Turning now to FIG. 5, the spacer 550 is depicted as having a circular cross-section. The bore 560 is located within the inner spacer 554. Further, there are gaps 562, 570 between the outer spacer 552 and the inner spacer 554. The dimensions of the gap would be the same as those discussed with respect to the embodiment shown in FIG. 3A. The embodiment of FIG. 3A can have a diameter that is the minor diameter of the embodiments shown in FIGS. 1A, 3A, and 4A.

Also, as will be appreciated by those in skill in the art, the outer spacer 552 can be movably mounted on the inner spacer 554 and the inner spacer 554 can be rotatably mounted on the shaft 102 of the implant 100 or any other suitable implant.

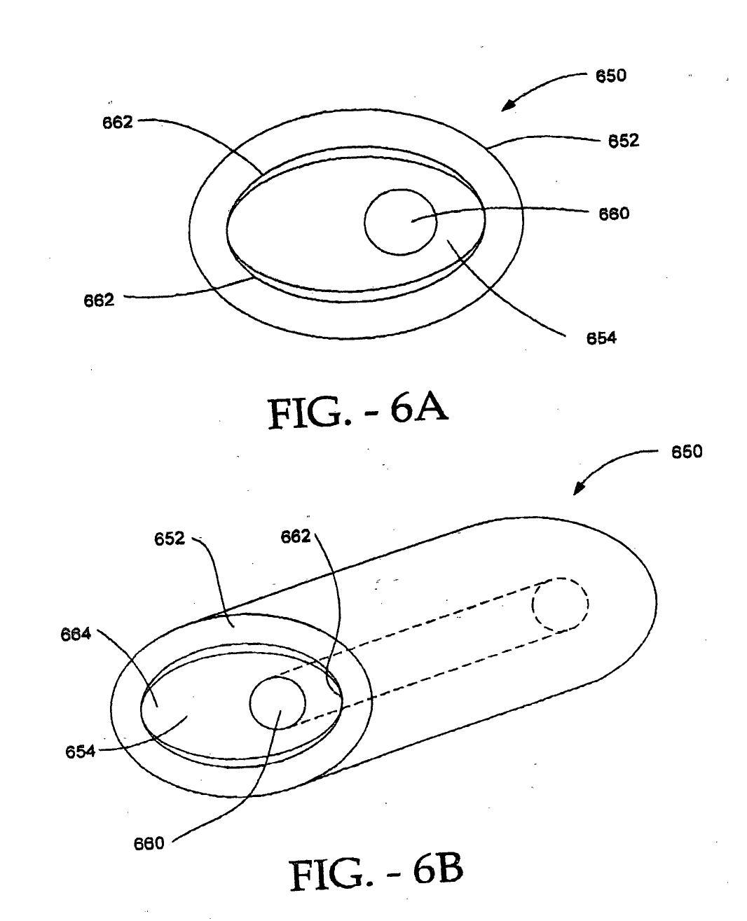

In FIGS. 6A and 6B, the spacer 650 is depicted as having an outer spacer 652 and an inner spacer 654 of two different cross-sectional shapes. In this embodiment, the outer spacer 652 is elliptical and the inner spacer is football-shaped in cross-sections. The bore 660 is located off-center within the inner spacer 654. However, as will be appreciated by those of skill in the art, the bore 660 can be located centrally within the inner spacer without departing from the scope of the invention.

The gaps 662 between the outer spacer 652 and the inner spacer 654 are crescent-shaped as a result of the inner and outer spacers having different cross-sectional shapes. Thus, the gap can have a width ranging from approximately between 0.25 mm at the minor diameter (greatest vertical height) to just enough space at the apexes 662, 664 of the inner spacer 654 so that the outer spacer can slide over the inner spacer. The inner spacer 654 can be rotatably mounted on the shaft 102 of the implant 100.

The embodiment of this implant as well as the several other implants described herein act to limit extension (backward bending) of the spine. These implants, however, do not inhibit the flexion (forward bending) of the spinal column.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and its equivalence.

1. An implant adapted to be placed between spinous processes comprising:
a body that includes a shaft;
a spacer rotatably mounted on the shaft; and
a tissue expander extending from the shaft;
wherein the tissue expander is at least in part radiolucent.

2. The implant of claim 1 wherein the tissue expander is selected from the group consisting of polyetheretherketone, polyetherketoneketone, polyaryletheretherketone, polyetherketone, polyetherketoneetherketoneketone, and polyetheretherketoneketone.

3. The implant of claim 1 wherein the spacer has a cross-sectional shape selected from the group consisting of elliptical-shaped, cylindrical-shaped, ovoid-shaped, oval-shaped, track-shaped, and rectangular-shaped with curved ends.

4. The implant of claim 1 wherein the spacer has a dimension selected from the group consisting of 6 mm, 8 mm, 10 m, 12 mm, and 14 mm.

5. The implant of claim 1 wherein the spacer has an off-center bore that receives the shaft so that the spacer can rotate about the shaft.

6. The implant of claim 1 wherein the tissue expander has a generally increasing cross-section from an end location to a location adjacent to the spacer.

7. The implant of claim 1 wherein the body includes a first wing extending from a location on the shaft on an opposite side of the spacer from which the tissue expander extends.

8. The implant of claim 1 wherein the shaft includes an attachment to which the tissue expander is affixed.

9. The implant of claim 8 wherein the attachment includes a device for receiving a wing.

10. The implant of claim 1 wherein the body includes a first wing extending from a location on the shaft on an opposite side of the spacer from which the tissue expander extends.

11. The implant of claim 10 wherein the body and the first wing are radiopaque such that under x-ray the implant resembles a T-shape.

12. The implant of claim 1 wherein the spacer is at least in part radiolucent.

13. The implant of claim 12 wherein at least one of the spacer and the tissues expander are selected from the group consisting of polyetheretherketone, polyetherketoneketone, polyaryletheretherketone, polyetherketone, polyetherketoneetherketoneketone, and polyetheretherketoneketone.

14. The implant of claim 1 further including:
a first wing located at one end of the shaft and a second wing located adjacent to the tissue expander such that the spacer is located between the first and the second wings,
wherein the body, the shaft, and the first and second wings are radiopaque and the tissue expander and spacer are radiolucent such that under imaging the implant resembles an H-shape.

15. The implant of claim 1 wherein the shaft includes an attachment to which the tissue expander is molded.

16. The implant or claim 15 wherein the attachment includes a device for receiving a wing.

17. The implant of claim 1 wherein the spacer includes:
an inner spacer that is rotatably mounted about the shaft; and
an outer spacer that is movably mounted on the inner spacer.

18. The implant of claim 17 wherein:
the inner spacer has one of flattened or slightly radiused upper and lower surfaces and rounded ends; and
the outer spacer has one of flattened or slightly radiused upper and lower surfaces and rounded ends.

19. An implant adapted to be placed between spinous processes comprising:
a body that includes a shaft; and
a spacer rotatably mounted on the shaft;
a tissue expander extending from the shaft;
wherein the tissue expander is at least in part radiolucent, and
wherein the spacer is at least in part radiolucent.

20. The implant of claim 19 including a wing located adjacent to the spacer.

21. The implant of claim 19 wherein at least one of the spacer and the tissues expander are selected from the group consisting of polyetheretherketone, polyetherketoneketone, polyaryletheretherketone, polyetherketone, polyetherketoneetherketoneketone, and polyetheretherketoneketone.

22. The implant of claim 19 wherein the tissue expander is selected from the group consisting of polyetheretherketone, polyetherketoneketone, polyaryletheretherketone, polyetherketone, polyetherketoneetherketoneketone, and polyetheretherketoneketone.

23. The implant of claim 19 wherein the tissue expander has a generally increasing cross-section from a distal end to a location adjacent to the spacer.

24. The implant of claim 19 wherein the implant has a first wing wherein the body and the first wing are radiopaque and the tissue expander and the spacer are radiolucent such that under imaging the implant resembles a T-shape.

25. The implant of claim 19 further including:
a first wing located at one end of the shaft and a second wing located adjacent to the tissue expander such that the spacer is located between the first and the second wings,
wherein the body, the shaft, and the first and second wings are radiopaque and the tissue expander and spacer are radiolucent such that under imaging the implant resembles an H-shape.

26. The implant of claim 19 wherein the spacer has a cross-sectional shape selected from the group consisting of elliptical-shaped, cylindrical-shaped, ovoid-shaped, oval-shaped, track-shaped, and rectangular-shaped with curved ends.

27. The implant of claim 19 wherein the spacer has a dimension selected from the group consisting of 6 mm, 8 mm, 10 m, 12 mm, and 14 mm.

28. The implant of claim 19 wherein the spacer has an off-center bore that receives the shaft so that the spacer can rotate about the shaft.

29. The implant of claim 19 wherein the spacer includes:
an inner spacer that is rotatably mounted about the shaft; and
an outer spacer that is movably mounted on the inner spacer.

30. The implant of claim 27 wherein:
the inner spacer has one of flattened or slightly radiused upper and lower surfaces and rounded ends; and
the outer spacer has one of flattened or slightly radiused upper and lower surfaces and rounded ends.

31. The implant of claim 19 wherein the body includes a first wing extending from a location on the shaft on an opposite side of the spacer from which the tissue expander extends.

32. The implant of claim 31 wherein the body and the first wing are radiopaque and the tissue expander and spacer are radiolucent such that under imaging the implant resembles a T-shape.

33. The implant of claim 19 wherein the shaft includes an attachment to which the tissue expander is affixed.

34. The implant of claim 33 wherein the attachment includes a device that can receive a wing.

35. The implant of claim 19 wherein the shaft includes an attachment to which the tissue expander is molded.

36. The implant or claim 35 wherein the attachment includes a device that can receive a wing.

37. An implant adapted to be placed between spinous processes comprising:
a body including a shaft;
a spacer rotatably mounted on the shaft; and
a tissue expander extending from the shaft;
wherein the tissue expander is at least in part selected from the group consisting of polyetheretherketone, polyetherketoneketone, and polyaryletheretherketone; and
wherein the spacer is at least in part selected from the group consisting of polyetheretherketone, polyetherketoneketone, and polyaryletheretherketone.

38. The implant of claim 37 further including:
a first wing located at one end of the shaft and a second wing located adjacent to the tissue expander such that the spacer is located between the first and the second wings,
wherein the body, the shaft, and the first and second wings are radiopaque such that under imaging the implant resembles an H-shape.

39. The implant of claim 37 wherein the shaft includes an attachment to which the tissue expander is molded.

40. The implant of claim 37 wherein the spacer has a cross-sectional shape selected from the group consisting of elliptical-shaped, cylindrical-shaped, ovoid-shaped, oval-shaped, track-shaped, and rectangular-shaped with curved ends.

41. The implant of claim 37 wherein the spacer has a dimension selected from the group consisting of 6 mm, 8 mm, 10 m, 12 mm, and 14 mm.

42. The implant of claim 37 wherein the spacer has an off-center bore that receives the shaft so that the spacer can rotate about the shaft.

43. The implant of claim 37 wherein the shaft includes an attachment to which the tissue expander is affixed.

44. The implant of claim 43 wherein the attachment includes a device for receiving a wing.

45. The implant of claim 37 wherein the spacer includes:
an inner spacer that is rotatably mounted about the shaft; and
an outer spacer that is movably mounted on the inner spacer.

46. The implant of claim 45 wherein:
the inner spacer has one of flattened or slightly radiused upper and lower surfaces and rounded ends; and
the outer spacer has one of flattened or slightly radiused upper and lower surfaces and rounded ends.

47. An implant adapted to be placed between spinous processes comprising:
a body includes a shaft;
a spacer rotatably mounted on the shaft;
a tissue expander extending from the shaft; and
wherein the tissue expander is at least in part selected from the group consisting of polyetheretherketone, polyetherketoneketone, polyaryletheretherketone, polyetherketone, polyetherketoneetherketoneketone, and polyetheretherketoneketone.

48. The implant of claim 47 wherein the spacer is at least in part selected from the group consisting of polyetheretherketone, polyetherketoneketone, polyaryletheretherketone, polyetherketone, polyetherketoneetherketoneketone, and polyetheretherketoneketone.

49. The implant of claim 37 wherein the body includes a first wing extending from a location on the shaft on an opposite side of the spacer from which the tissue expander extends.

50. The implant of claim 47 wherein the tissue expander has a generally increasing cross-section from a distal end to a location adjacent to the spacer.

51. The implant of claim 49 wherein the body and the first wing are radiopaque such that under imaging the implant resembles a T-shape.

52. The implant of claim 48 further including:
a first wing located at one end of the shaft and a second wing located adjacent to the tissue expander such that the spacer is located between the first and the second wings,
wherein the body, the shaft, and the first and second wings are radiopaque such that under imaging the implant resembles an H-shape.

53. The implant of claim 47 wherein the shaft includes an attachment to which the tissue expander is affixed.

54. The implant of claim 47 wherein the spacer has a dimension selected from the group consisting of 6 mm, 8 mm, 10 m, 12 mm, and 14 mm.

55. The implant of claim 47 wherein the spacer has a cross-sectional shape selected from the group consisting of elliptical-shaped, cylindrical-shaped, ovoid-shaped, oval-shaped, track-shaped, and rectangular-shaped with curved ends.

56. The implant of claim 47 wherein the spacer has an off-center bore that receives the shaft so that the spacer can rotate about the shaft.

57. The implant of claim 47 wherein the shaft includes an attachment to which the tissue expander is molded.

58. The implant of claim 57 wherein the attachment includes a device for receiving a wing.

59. The implant or claim 58 wherein the attachment includes a device for receiving a wing.

60. The implant of claim 47 wherein the spacer includes:
an inner spacer that is rotatably mounted about the shaft; and
an outer spacer that is movably mounted on the inner spacer.

61. The implant of claim 60 wherein:
the inner spacer has one of flattened or slightly radiused upper and lower surfaces and rounded ends; and
the outer spacer has one of flattened or slightly radiused upper and lower surfaces and rounded ends.

62. An implant adapted to be placed between spinous processes comprising:
a body having a shaft extending therefrom;
a spacer rotatably mounted on the shaft; and
a tissue expander extending from the shaft,
wherein the body and the shaft are radiopaque, and further wherein the spacer and the tissue expander are radiolucent.

63. The implant of claim 62 wherein the spacer and tissue expander are selected from the group consisting of polyetheretherketone and polyetherketoneketone.

64. The implant of claim 62 wherein the spacer is comprised of:
an inner spacer that is rotatably mounted about the shaft; and
an outer spacer that is movably mounted relative to the inner spacer.

65. The implant of claim 62 wherein:
the inner spacer has one of a flattened or a slightly radiused upper and lower surfaces and rounded first and second end; and
the outer spacer has one of a flattened or a slightly radiused upper and lower surfaces and rounded first and second ends.

66. The implant of claim 64 wherein the inner spacer and the outer spacer are selected from the group consisting of polyetheretherketone, polyetherketoneketone, and polyaryletheretherketone.

67. The implant of claim 62 further comprising a first and second wing, wherein the wings are located at opposite ends of the spacer and wherein the body, shaft and wings are a radiopaque “H” on imaging film.

68. A method of locating an implant relative to spinous processes of vertebrae comprising the steps of:
implanting an implant that has first and second wings connected by a shaft that are radiopaque and with a spacer located between the first and second wings and a tissue expander extending from the shaft that are radiolucent;
locating the implant either during the implantation step or after the implantation step using an imaging technique which identifies the implant by an “H” pattern.

69. The method of locating the implant of claim 68 wherein the “H” pattern shows the first and second wings being substantially parallel and rail-like and the shaft being perpendicular to the first and second wings.

70. A method of locating an implant relative to spinous processes of vertebrae comprising the steps of:
implanting an implant that has a first wing connected to a shaft that are radiopaque and with a spacer located adjacent the first wing and a tissue expander extending from the shaft that are radiolucent;
locating the implant either during the implantation step or after the implantation step using an imaging technique which identifies the implant by an “T” pattern.

71. The method of locating the implant of claim 68 wherein the “T” pattern shows the first and wing being rail-like and the shaft being perpendicular to the first wing.
